(19) United States
(12) Patent Application Publication
(10) Pub. No.: US 2023/0369927 A1
(43) Pub. Date: Nov. 16, 2023

(54) ELECTROMAGNETIC ROTATING DEVICE

(71) Applicant: Yu-Chu CHOU, Kaohsiung City (R.O.C.) (TW)

(72) Inventor: Yu-lun HUANG, Kaohsiung City (R.O.C.) (TW)

(73) Assignee: Yu-Chu CHOU, Kaohsiung City (R.O.C.) (TW)

(21) Appl. No.: 18/142,610

(22) Filed: May 3, 2023

(30) Foreign Application Priority Data
May 12, 2022 (TW) 111117933

(51) Int. Cl. H02K 1/2795; H02K 21/24; H02K 3/28; A61M 60/104

(52) U.S. Cl. CPC H02K 1/2795; H02K 21/24; H02K 3/28; A61M 60/104

(57) ABSTRACT

An electromagnetic rotating device is provided. The electromagnetic rotating device includes a base, a pole disc, and a plurality of pole control units. Magnetic elements of the pole disc are arranged on a disc body. A second circular orbit of the pole disc is disposed on a lower surface of the disc body. The second circular orbit is configured to be slidably combined on a first circular orbit of the base. The pole control units are arranged on the base and configured to magnetically drive the magnetic elements to move, so that the pole disc rotates relative to the base.

### CROSS REFERENCE TO RELATED APPLICATIONS

This application claims the priority benefit of Taiwan Patent Application No. 111117933, filed on May 12, 2022. The contents of which are herein incorporated by reference.

### FIELD OF INVENTION

Embodiments of the present disclosure relate generally to a rotating device, in particular to an electromagnetic rotating device.

### BACKGROUND OF INVENTION

Generally, blood circulation or blood flow in a body directly or indirectly affects the health of that body. For example, if a person has poor blood circulation in the gastrointestinal tract, the person has a digestive disorder; if the person has poor or inefficient blood circulation in a limb, the person experiences pain in the limb; if the person has poor or irregular blood circulation in the joints, such as the knees, the person suffers from arthritis; if the person has poor blood circulation in the heart, the person develops a cardiac disorder; if the person has poor or weak blood circulation to the head, the person experiences headaches or reduced brain function; if the person has poor blood circulation in the lungs, the person suffers from lung disease. Also, serious problems with blood circulation may lead to cerebral infarction, cerebral hemorrhage, or cerebral hemorrhage. When blood does not circulate smoothly through a part of the body, oxygen and nutrients are not adequately supplied to said part. The waste generated by metabolism cannot be properly excreted. As a result, the person experiences pain, suffering, or disease.

Recently, pain or other afflictions have been treated by using magnetic fields to improve blood circulation. For example, some traditional methods teach treating diseases and inflammations of the body by attaching a monopolar magnet to the body to generate a monopolar magnetic field. Moreover, conventional devices provide N poles and S poles on opposite sides. Pain or disease is treated by keeping the surface of the N pole in contact with the first body part and the surface of the S pole in contact with the second body part. However, conventional devices cannot effectively generate magnetic field waves, and they are not easy to carry and operate.

Therefore, it is necessary to provide an improved electromagnetic rotating device to solve the problems existing in the conventional technologies, as described above.

### SUMMARY OF INVENTION

An object of the present disclosure is to provide an electromagnetic rotating device that uses pole control units to drive the rotation of magnetic elements and generate variable magnetic field waves. This makes it possible to easily stimulate and disturb proteins through the induced magnetic fields, while also improving blood flow through the use of these magnetic field waves.

To achieve the above object, the present disclosure provides an electromagnetic rotating device, which comprises a base, a pole disc, and a plurality of pole control units. The base comprises a body and a first circular orbit, wherein the first circular orbit is arranged on an upper surface of the body. The pole disc comprises a disc body, a plurality of magnetic elements, and a second circular orbit, wherein the magnetic elements are disposed on the disc body, the second circular orbit is arranged on a lower surface of the disc body and located at the periphery of the magnetic elements, and the second circular orbit is configured to be slidably assembled on the first circular orbit. The plurality of pole control units are disposed on the base, wherein the pole control units are configured magnetically drive the magnetic elements to move, so that the pole disc is rotated relative to the base.

According to an embodiment of the present disclosure, the pole control units are located between the base and the pole disc.

According to an embodiment of the present disclosure, the pole control units comprises a top coil layer, a plurality of pole coil layers, at least one connecting coil layer, and a bottom coil layer stacked in sequence, the connecting coil layer is arranged between the two pole coil layers.

According to an embodiment of the present disclosure, the corresponding pole control unit is formed by electrically connecting one of the top coils of the top coil layer, one of the pole coils of the plurality of pole coil layers, one of the connecting coils of the connecting coil layer, and one of the bottom coils of the bottom coil layer.

According to an embodiment of the present disclosure, one of the pole coils of the plurality of pole coil layers is wound into a triangle or a circle.

According to an embodiment of the present disclosure, a coil hole is formed on the body of the base, the coil hole is configured to allow the top coils of the top coils layer and the bottom coils of the bottom coil layer to pass through.

According to an embodiment of the present disclosure, the pole control units are arranged at intervals on an upper surface of the base and located outside the first circular orbit.

According to an embodiment of the present disclosure, each of the pole control units includes a front plate, a rear plate, a connecting plate, and a coil structure, the connecting plate is connected between the front plate and the rear plate, and the coil structure is formed on the connecting plate.

According to an embodiment of the present disclosure, the magnetic elements are equidistantly embedded in the disk body, and the magnetic poles of the two adjacent magnetic elements are different.

According to an embodiment of the present disclosure, a first circular hole is formed on the body of the base, a second circular hole is formed on the disk body of the pole disc, and the magnetic elements are located at the periphery of the second circular hole.

As above described, the electromagnetic rotating device is configured to be attached to the skin, design using the pole control units to drive the rotation of the magnetic elements, so that the electromagnetic rotating device can generate the variable magnetic field wave. Proteins are easily stimulated and disturbed by the magnetic field induced by the changing magnetic field wave. At the same time, the magnetic field wave can also improve blood flow, thereby achieving the effect of promoting blood circulation. Furthermore, the electromagnetic rotating device is only composed of the base, the pole disc, and the pole control units, or is composed of pole control units alone, which has the advantages of simple structure, easy portability, and convenient operation.

### DESCRIPTION OF DRAWINGS

FIG. 1 is a perspective view according to an embodiment of an electromagnetic rotating device of the present disclosure.

FIG. 2 is a cross-sectional view according to an embodiment of an electromagnetic rotating device of the present disclosure.

FIG. 3 is a perspective view of pole control units according to an embodiment of the electromagnetic rotating device of the present disclosure.

FIG. 4 is a top view of the pole control units according to an embodiment of the electromagnetic rotating device of the present disclosure.

FIG. 5 is a top view of the pole control units showing a 360-degree magnetic field according to an embodiment of the electromagnetic rotating device of the present disclosure.

FIG. 6 is a top view of the pole control units showing a 180-degree magnetic field according to an embodiment of the electromagnetic rotating device of the present disclosure.

FIG. 7 is a top view of the pole control units showing a 90-degree magnetic field according to an embodiment of the electromagnetic rotating device of the present disclosure.

FIG. 8 is a top view of the pole control units showing a magnetic field with an unequal proportion according to an embodiment of the electromagnetic rotating device of the present disclosure.

FIG. 9 is a schematic diagram of magnetic field wave generated by the pole disc of the pole control unit according to an embodiment of the electromagnetic rotating device of the present disclosure.

FIG. 10 is a top view of another aspect of the pole coils of the pole control unit according to an embodiment of the electromagnetic rotating device of the present disclosure.

FIG. 11 is a perspective view according to another embodiment of the electromagnetic rotating device of the present disclosure.

FIG. 12 is a cross-sectional view according to another embodiment of the electromagnetic rotating device of the present disclosure.

FIG. 13 is a top view of a pole control unit according to another embodiment of the electromagnetic rotating device of the present disclosure.

### DETAILED DESCRIPTION OF PREFERRED EMBODIMENTS

The structure and the technical means adopted by the present disclosure to achieve the above and other objects can be best understood by referring to the following detailed description of the preferred embodiments and the accompanying drawings. Furthermore, directional terms described by the present disclosure, such as upper, lower, front, back, left, right, inner, outer, side, longitudinal/vertical, transverse/horizontal, etc., are only directions by referring to the accompanying drawings, and thus the used directional terms are used to describe and understand the present disclosure, but the present disclosure is not limited thereto.

Refer to FIG. 1, an embodiment of the electromagnetic rotating device of the present disclosure is illustrated. The electromagnetic rotating device comprises a base 2, a pole disc 3, and a plurality of pole control units 4. The electromagnetic rotating device is configured to be attached to the skin. The detailed structure of each component, assembly relationships, and principles of operation for the present disclosure will be described in detail hereinafter.

Refer to FIG. 1, the base 2 comprises a body 21 and a first circular orbit 22, wherein the first circular orbit 22 is arranged on an upper surface of the body 21, a first circular hole 23 is formed on the body 21 of the base 2, and the first circular hole 23 is located at a geometric center of the body 21.

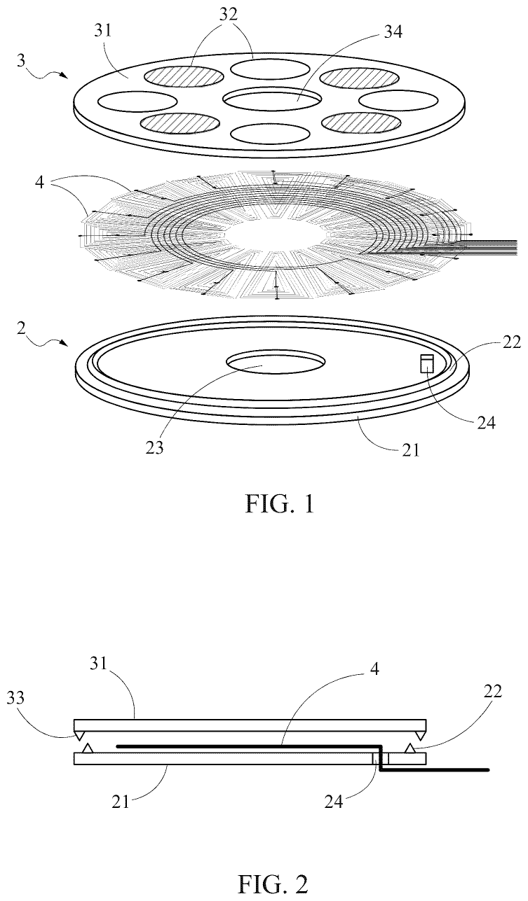

Refer to FIG. 1 and FIG. 2, the pole disc 3 comprises a disc body 31, a plurality of magnetic elements 32, and a second circular orbit 33, wherein the magnetic elements 32 are disposed on the disc body 31, the second circular orbit 33 is arranged on a lower surface of the disc body 31, the second circular orbit 33 located at the periphery of the magnetic elements 32, and the second circular orbit 33 is configured to be slidably assembled on the first circular orbit 22. Specifically, a second circular hole 34 is formed on the disk body 31 of the pole disc 3, the first circular hole 23 corresponds to the second circular hole 34, and the magnetic elements 32 are located at the periphery of the second circular hole 34. In the embodiment, the magnetic elements 32 are magnets.

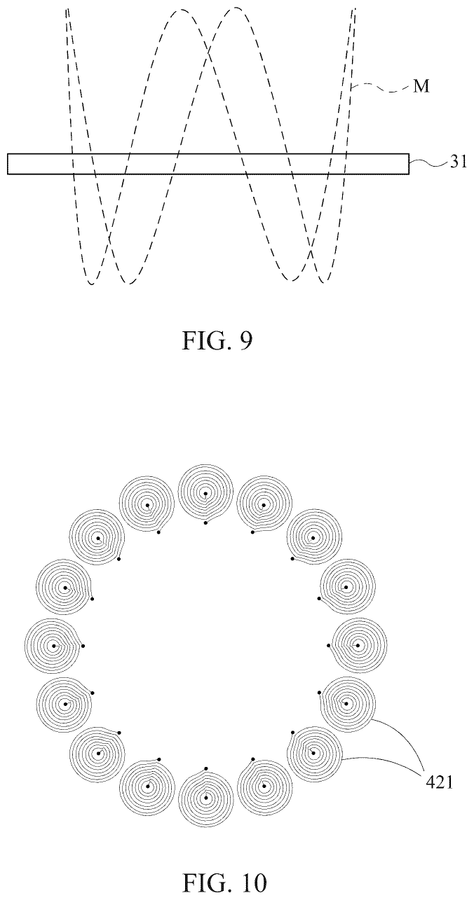

Refer to FIG. 1 and FIG. 2, the plurality of pole control units 4 are disposed on the base 2, wherein the pole control units 4 are configured magnetically drive the magnetic elements 32 to move. The pole disc 3 can rotate relative to the base 2, so that the electromagnetic rotating device generates a variable magnetic field wave M, such as a sine wave (see FIG. 9). In the embodiment, the pole control units 4 are located between the base 2 and the pole disc 3.

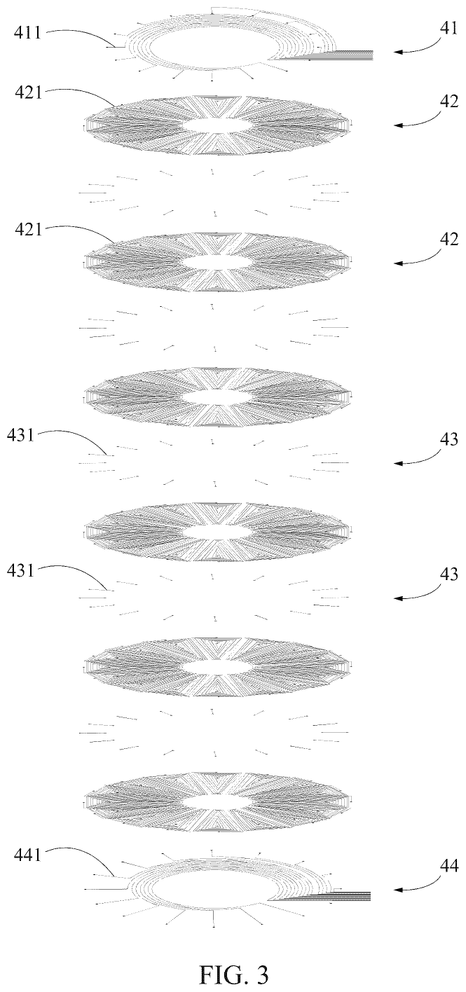

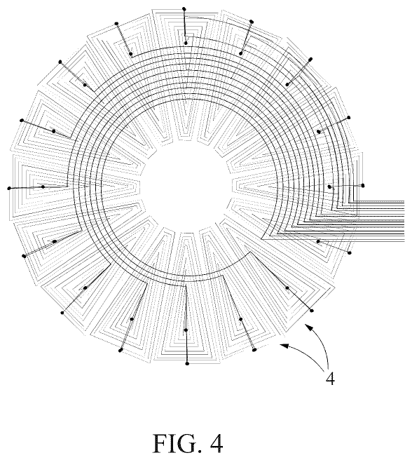

Refer to FIG. 1 and FIG. 3, the pole control units 4 (see FIG. 1) comprises a top coil layer 41, a plurality of pole coil layers 42, at least one connecting coil layer 43, and a bottom coil layer 44 stacked in sequence (see FIG. 3), the connecting coil layer 43 is arranged between the two pole coil layers 42. Specifically, this embodiment is provided with 6 layers of pole coil layers 42 and 5 layers of connecting coil layer 43. The corresponding pole control unit 4 is formed by electrically connecting one of the top coils 411 of the top coil layer 41, one of the pole coils 421 of the plurality of pole coil layers 42, one of the connecting coils 431 of the connecting coil layer 43, and one of the bottom coils 441 of the bottom coil layer 44. In the embodiment, one of the pole coils 421 of the plurality of pole coil layers 42 may be wound into a triangle (see FIG. 4) or a circle (see FIG. 10).

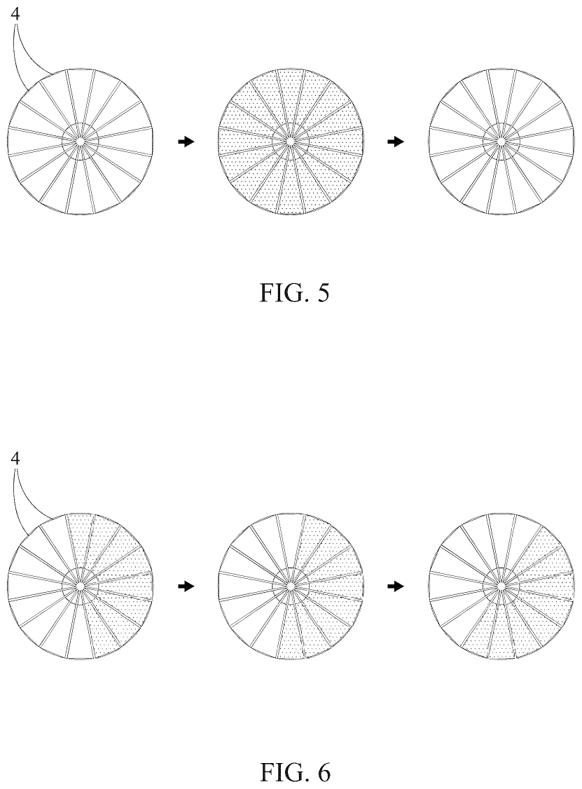

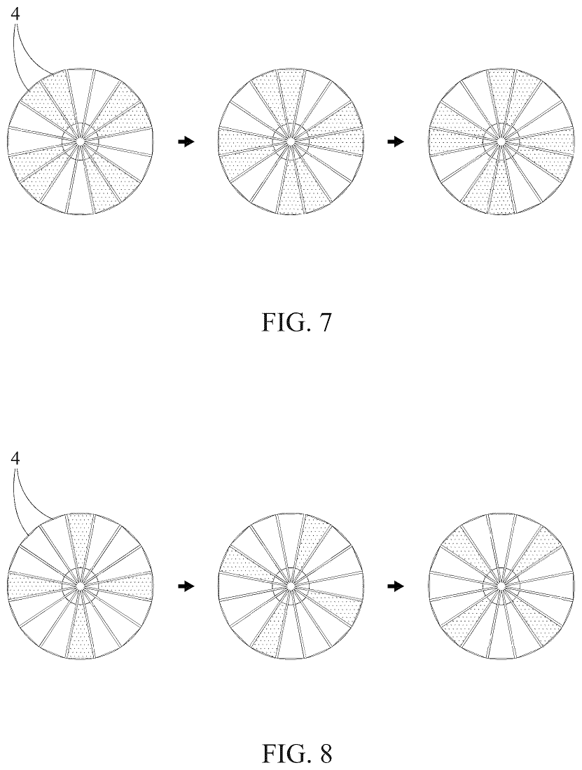

Further, each of the pole coil layers 42 in the embodiment has 16 triangular pole coils 421, and a circle is formed by 16 triangular pole coils 421 (see FIG. 4). As shown in FIG. 4 to FIG. 8, the magnetic fields of the pole control units 4 can be used independently, wherein various forms of magnetic poles are derived by controlling the current of multiple coils. For example, a 360-degree magnetic field, that is, the 16 pole coils 421 are all controlled to be the same magnetic pole (see FIG. 5); a 180-degree magnetic field, that is, the pole coils 421 are controlled to distinguish two kinds of magnetic poles at 180 degrees in polar coordinate (see FIG. 6); 90-degree magnetic field, that is, the pole coils 421 are controlled to distinguish different staggered magnetic poles at 90 degrees in polar coordinate (see FIG. 7); the magnetic field with unequal proportions, that is, the pole coils 421 are controlled to interleave different poles in unequal proportions (see FIG. 8).

Refer to FIG. 1, FIG. 2, and FIG. 3, a coil hole 24 is formed on the body 21 of the base 2, the coil hole 24 is configured to allow the top coils 411 of the top coils layer 41 and the bottom coils 441 of the bottom coil layer 44 to pass through. Furthermore, the magnetic elements 32 are equidistantly embedded in the disk body 31, and the magnetic poles of the two adjacent magnetic elements 32 are different.

According to the above structure, the pole control units 4 can be controlled to switch different magnetic poles, the magnetic elements 32 are moved by means of magnetic force, and then the pole disc 3 is driven to rotate relative to the base 2. The magnetic elements 32 are used to rotate along the axis of the disk body 31 to generate a variable magnetic field wave M.

As above described, the electromagnetic rotating device is configured to be attached to the skin, design using the pole control units 4 to drive the rotation of the magnetic elements 32, so that the electromagnetic rotating device can generate the variable magnetic field wave M. Proteins are easily stimulated and disturbed by the magnetic field induced by the changing magnetic field wave M. At the same time, the magnetic field wave M can also improve blood flow, thereby achieving the effect of promoting blood circulation. Furthermore, the electromagnetic rotating device is only composed of the base 2, the pole disc 3 and the pole control units 4, or is composed of pole control units 4 alone, which has the advantages of simple structure, easy portability, and convenient operation.

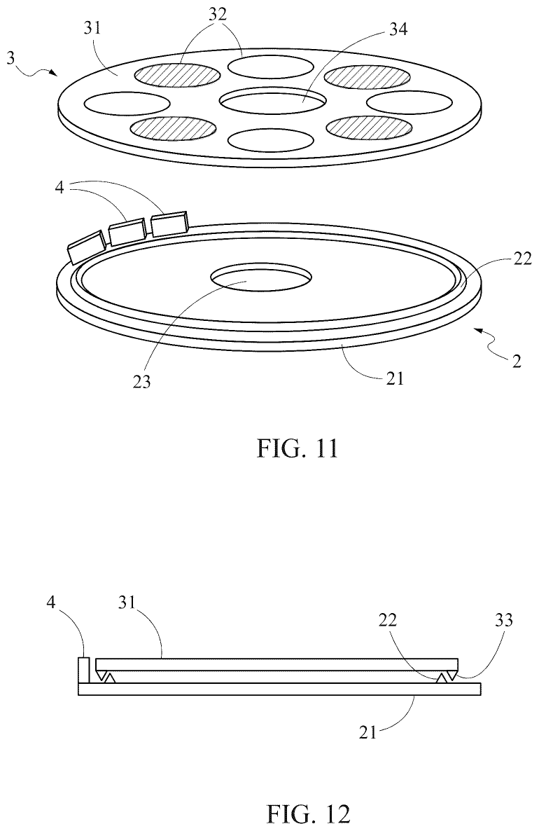

Refer to FIG. 11 and FIG. 12, another embodiment of the electromagnetic rotating device of the present disclosure is illustrated. The embodiment is similar to the above-mentioned embodiment of the present disclosure, and generally uses the same component names and figure numbers, wherein the difference of the electromagnetic rotating device of the present disclosure is that the pole control units 4 (e.g., 3 or more) are arranged at intervals on an upper surface of the body 21 of the base 2 and the pole control units 4 are located outside the first circular orbit.

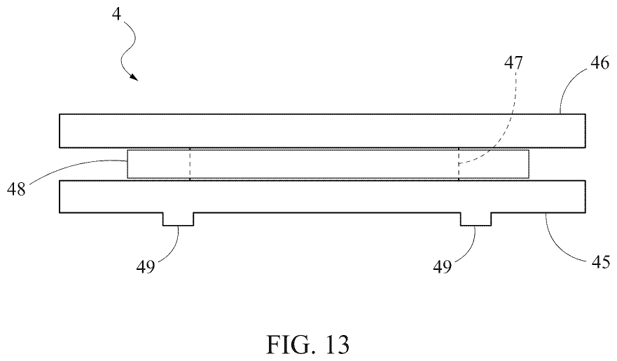

Refer to FIG. 11 and FIG. 13, further, each of the pole control units 4 includes a front plate 45, a rear plate 46, a connecting plate 47, and a coil structure 48, the connecting plate 47 is connected between the front plate 45 and the rear plate 46, and the coil structure 48 is formed on the connecting plate 47. In the embodiment, the coil structure 48 is a coil made of enameled wire wound on the connecting plate 47. In another embodiment, the coil structure 48 is a coil formed by evaporating several layers of metal and insulating layers respectively. Moreover, each of the magnetic pole control units 4 further includes two bumps 49. The bumps 49 are formed on a surface of the front plate 45 facing the pole disc 3 to enhance the magnetic induction effect of the pole control units 4 on the magnetic elements 32.

As above described, the electromagnetic rotating device is configured to be attached to the skin, design using the pole control units 4 to drive the rotation of the magnetic elements 32, so that the electromagnetic rotating device can generate the variable magnetic field wave M. Proteins are easily stimulated and disturbed by the magnetic field induced by the changing magnetic field wave M. At the same time, the magnetic field wave M can also improve blood flow, thereby achieving the effect of promoting blood circulation.

Although the present disclosure has been disclosed in preferred embodiments, it is not intended to limit the present invention. Anyone who is familiar with this technique may make various changes and modifications without departing from the spirit and scope of the present disclosure. Therefore, the scope of protection of the present disclosure shall be subject to the definition of the attached patent scope.

What is claimed is:

1. An electromagnetic rotating device, comprising:
a base comprising a body and a first circular orbit, wherein the first circular orbit is arranged on an upper surface of the body;
a pole disc comprising a disc body, a plurality of magnetic elements, and a second circular orbit, wherein the magnetic elements are disposed on the disc body, the second circular orbit is arranged on a lower surface of the disc body and located at the periphery of the magnetic elements, and the second circular orbit is configured to be slidably assembled on the first circular orbit; and
a plurality of pole control units disposed on the base, wherein the pole control units are configured magnetically drive the magnetic elements to move, so that the pole disc is rotated relative to the base.

2. The electromagnetic rotating device according to claim 1, wherein the pole control units are located between the base and the pole disc.

3. The electromagnetic rotating device according to claim 2, wherein the pole control units comprises a top coil layer, a plurality of pole coil layers, at least one connecting coil layer, and a bottom coil layer stacked in sequence, the connecting coil layer is arranged between the two pole coil layers.

4. The electromagnetic rotating device according to claim 3, wherein the corresponding pole control unit is formed by electrically connecting one of the top coils of the top coil layer, one of the pole coils of the plurality of pole coil layers, one of the connecting coils of the connecting coil layer, and one of the bottom coils of the bottom coil layer.

5. The electromagnetic rotating device according to claim 4, wherein one of the pole coils of the plurality of pole coil layers is wound into a triangle or a circle.

6. The electromagnetic rotating device according to claim 4, wherein a coil hole is formed on the body of the base, the coil hole is configured to allow the top coils of the top coils layer and the bottom coils of the bottom coil layer to pass through.

7. The electromagnetic rotating device according to claim 1, wherein the pole control units are arranged at intervals on an upper surface of the base and located outside the first circular orbit.

8. The electromagnetic rotating device according to claim 7, wherein each of the pole control units includes a front plate, a rear plate, a connecting plate, and a coil structure, the connecting plate is connected between the front plate and the rear plate, and the coil structure is formed on the connecting plate.

9. The electromagnetic rotating device according to claim 1, wherein the magnetic elements are equidistantly embedded in the disk body, and the magnetic poles of the two adjacent magnetic elements are different.

10. The electromagnetic rotating device according to claim 1, wherein a first circular hole is formed on the body of the base, a second circular hole is formed on the disk body of the pole disc, and the magnetic elements are located at the periphery of the second circular hole.
